(19) United States
(12) Patent Application Publication
(10) Pub. No.: US 2002/0133002 A1
(43) Pub. Date: Sep. 19, 2002

(54) COMPUTER IMPLEMENTED NUCLEIC ACID ISOLATION METHOD AND APPARATUS

(76) Inventors: ELLEN M. HEATH, MINNETONKA, MN (US); RUTH SHUMAN, MINNETONKA, MN (US)

Correspondence Address: FOGG SLIFER & POLGLAZE, P.A., P.O. BOX 581009, MINNEAPOLIS, MN, 55458-1009, US

(21) Appl. No.: 09/361,829

(22) Filed: Jul. 27, 1999

Related U.S. Application Data
(63) Continuation-in-part of application No. 09/255,146, filed on Feb. 22, 1999.

(51) Int. Cl. C07H 21/00; B01J 8/00; G06G 7/48

(52) U.S. Cl. 536/25.4; 422/129; 703/11

(57) ABSTRACT

A computer program module and computer system for issuing controls to an automated DNA isolation apparatus includes a series of sub-program modules for controlling the operation of generic processes of DNA isolation. The sub-modules may be used to construct an automated DNA isolation protocol specific to the user's purpose. In other embodiments, a computer program module and computer system issue controls to an automated nucleic acids isolation apparatus including subprogram modules for controlling nucleic acid isolation functions.

This application is a continuation in part of U.S. patent application Ser. No. 09/255,146, filed Feb. 22, 1999, pending.

### FIELD

The present invention relates generally to isolation of deoxyribonucleic acid (DNA), and more specifically to a method for automating the isolation of DNA. The present invention also relates to a method of automated isolation of nucleic acids.

### BACKGROUND

As medical science continues to advance, the uses for DNA and the desire for increased quantities of isolated DNA have led to several methods for the isolation of DNA. Isolation of DNA is an important process used in numerous applications, including diagnosis of certain infections, forensic sciences, and other clinical applications, as well as recombinant DNA research, cloning, sequencing and the like.

The isolation of DNA from biological samples has been and continues to be labor intensive, requiring time consuming and repetitive tasks that occupy a technician, often to the exclusion of other tasks. The repetitive yet delicate process steps of DNA isolation require precision and attention to detail, and may often rely on the skill of the technician responsible for the isolation. Repetitive application of precise process steps lends itself to errors which may negatively affect the quality and/or quantity of DNA isolated from a sample. In the case of unique or limited samples, such errors may occur when dealing with samples that cannot be duplicated, or are irreplaceable.

Further, many processes used in DNA isolation involve the use of toxic, caustic, poisonous, or otherwise dangerous chemicals, as well as equipment that may be extremely delicate and expensive. Great care must be used by a technician to avoid damaging equipment, and to avoid the harm that may result from contact with hazardous materials.

The process of DNA isolation also lends itself to contamination, and great care must be taken to protect against contamination. The generally large number of process steps required to isolate DNA increase the risk for contamination and cross-contamination of samples.

DNA sample materials must be handled with care not only because of the risk of contact with hazardous materials and the risk of contamination of the sample, but also because the samples are typically fragile. Coagulated DNA is in the form of strands suspended in a liquid. Viscous effects in the liquid can tear DNA strands. Handling of samples is therefore also subject to careful consideration.

Currently, manual processes for isolation of DNA require a time intensive operation of one (1) to 24 hours, including an overnight incubation period. Excluding any incubation period, a technician may be required to perform upward of twenty tasks on a regular basis during the isolation process. Human interaction with the process steps is intensive. It is difficult for a technician or other operator to accomplish much else during the short intervals of idle time between required human interaction with the materials in the manual process.

In DNA isolation, a sample of biological material is typically placed in a sample vessel, and the processes comprising the DNA isolation process are performed on the tube and its contents. Materials may be removed from the vessel, transferred to another tube, and the like. Procedures for the setup of DNA isolation processes are known in the art and will not be described further herein.

### SUMMARY

The present invention overcomes the problems of the prior art by providing methods for controlling the automated isolation of DNA, specifically by computerizing the process by which a DNA isolation apparatus may be controlled. A further embodiment comprises a driver program for issuing commands to a machine capable of performing the functions required in isolating DNA.

A computer implemented method for controlling the operation of a machine for isolating DNA reduces the time required for a technician to be present and occupied with the DNA isolation process, freeing the technician to perform other tasks while DNA isolation is accomplished. Further, such a controlled automation process allows the precise and accurate repetition over a multiplicity of iterations of a method, ensuring quality control for the isolation process.

In one embodiment, a program module having various sub-modules allows for the creation of a customized command set for the control of an apparatus for isolating DNA. In such an embodiment, the sub-modules could be variably programmed and sequenced to provide a method for creating a computer controlled command set for DNA isolation by an automated apparatus. Such an apparatus could include a stand-alone apparatus, a robotic workstation, or the like.

Robotics to reduce the possibility for human error and for human contamination would be important step. With a robotic embodiment run by computer software, it would reduce the likelihood that the samples being worked on would be contaminated by accidental human contact. Human interaction with the actual samples would be reduced by automation, and therefore, the risk of contamination would also be reduced.

### BRIEF DESCRIPTION OF THE DRAWINGS

FIG. 1 is a flow chart diagram of a method of isolating DNA;

FIG. 2 is a flow chart diagram of a more detailed method of isolating DNA;

FIG. 3 is a block diagram of an embodiment of the present invention;

FIG. 3a is a block diagram of another embodiment of the present invention;

FIG. 4a is a view of a representative screen of a graphical user interface according to an embodiment of the present invention;

FIGS. 4b, 4c, 4d, and 4e are views of other representative screens of a graphical user interface according to an embodiment of the invention;

FIGS. 4f, 4g, 4h, 4i, and 4j are views of other representative screens of a graphical user interface according to another embodiment of the invention;

FIG. 5 is a flow chart diagram of a method embodiment of the present invention; and

FIG. 6 is a diagram of a computer in which embodiments of the present invention may be implemented.

### DESCRIPTION OF EMBODIMENTS

In the following detailed description of the embodiments, reference is made to the accompanying drawings which form a part hereof, and in which is shown by way of illustration specific embodiments in which the invention may be practiced. It is to be understood that other embodiments may be utilized and structural changes may be made without departing from the scope of the present invention.

Some portions of the detailed descriptions which follow are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like. It should be borne in mind, however, that all of these and similar terms are to be associated with the appropriate physical quantities and are merely convenient labels applied to these quantities. Unless specifically stated otherwise as apparent from the following discussions, it is appreciated that throughout the present invention, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

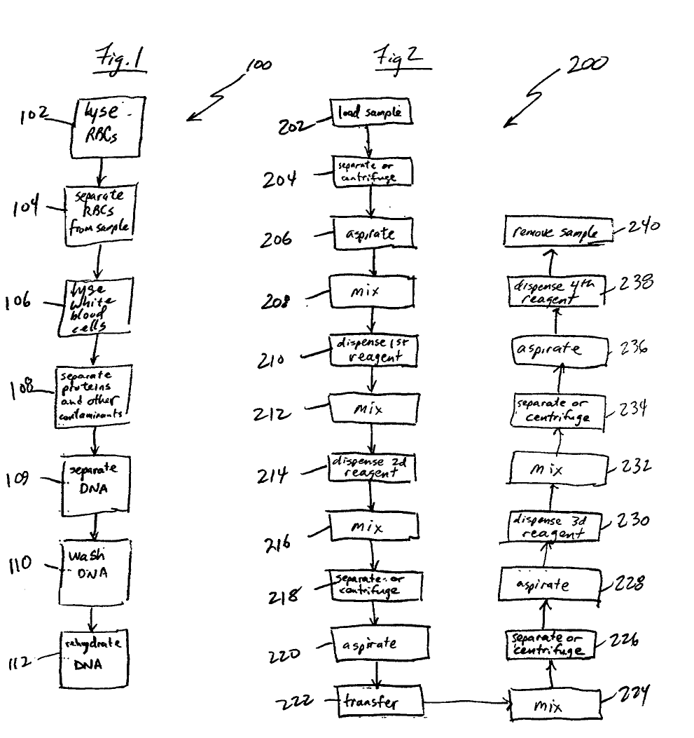

A general method 100 of isolating DNA is shown in FIG. 1 to comprise lysing red blood cells (RBCs) in block 102, separating RBCs from blood in block 104, lysing white blood cells in block 106, separating proteins and other contaminants from DNA in block 108, separating DNA in block 109, washing the DNA in block 110, and rehydrating the DNA in block 112. Such a process may involve a number of further process steps specific to the specific DNA isolation process involved.

A more specific process 200 for isolating DNA is shown in FIG. 2 to comprise loading a sample from which DNA is to be isolated into a centrifuge in block 202, separating or centrifuging the sample for a predetermined time at a predetermined g force in block 204, aspirating excess supernatant in block 206, mixing to resuspend a pellet in block 208, dispensing a predetermined amount of a first reagent in block 210, mixing in block 212, dispensing a predetermined amount of a second reagent in block 214, mixing in block 216, separating or centrifuging for a predetermined amount of time at a predetermined g-force in block 218, and aspirating excess supernatant in block 220. After aspiration in block 220, process flow continues with transferring the sample to a second tube containing a predetermined volume of fluid in block 222, mixing in block 224, separating or centrifuging for a predetermined time and at a predetermined g-force in block 226, aspirating excess supernatant in block 228, dispensing a predetermined amount of a third reagent in block 230, mixing in block 232, separating or centrifuging for a predetermined time and at a predetermined g-force in block 234, aspirating a predetermined amount of excess supernatant in block 236, and dispensing a predetermined amount of a fourth reagent in block 238. The resultant sample may be stored as is appropriate in block 240. It should be understood that further reagents may be required for different processes.

Even more specifically, the method 200 described above may have specific parameters for each operation. In one embodiment, the method 200 has parameters as follows. Centrifuging in blocks 204 and 218 is undertaken for a period often (10) minutes at 2,000 g, centrifuging in block 226 is undertaken for a time period of three (3) minutes at 2,000 g, and centrifuging in block 234 is undertaken for a time period of one (1) minute at 2,000 g. It should be understood that the time period required for adequate separation by centrifuging may vary with the centrifuge force. At higher g-forces, less time may be required. For example, centrifugation time is inversely proportional to the square of the rotational speed. This means that by doubling the rotational speed the time to collect the precipitate in the bottom of the tube is reduced to one-fourth the original precipitation time.

Aspirating in block 206 comprises removal of approximately 40 ml of supernatant, aspirating in block 220 comprises removing approximately 22 ml of supernatant, and aspirating in block 236 comprises removing approximately ten (10) ml of volume. Aspiration may be accomplished at different rates depending upon the desired result of the aspiration. It should also be understood that the process by which supernatant is removed may be varied, and that measurement of the volume may be by any known method, including but not limited to volume, weight, and the like, or that excess supernatant may be removed with remaining volume or mass as a determining factor. Such processes might include optically sensing the remaining material in the sample or the like.

Mixing is accomplished at various levels from gentle to vigorous. Mixing may be accomplished by any number of processes including physical agitation and a combination of aspirating and dispensing. Aspiration mixing is achieved by a cycle of aspirating and dispensing of fluid in and out of a pipette. Increasing and decreasing the aspiration and dispensation rates and volumes varies the mixing intensity. Mixing in block 208 comprises aspirating and dispensing approximately one (1) ml three (3) times, mixing in blocks 212, 216, and 224 comprises aspirating and dispensing approximately ten (10) ml three (3) times, and mixing in block 232 comprises aspirating and dispensing approximately ten (10) ml five (5) times.

While the process flow described above is representative of one process for isolating DNA from a sample, in this case a sample of mammalian blood, other biological samples follow different process flow. The base processes of centrifugation, aspiration, mixing, and dispensing remain substantially the same. However, the parameters for the process flow may change due to a number of factors. For example, the specific volume details given above are for a sample of 10 ml mammalian whole blood in a 50 ml centrifuge tube. If the quantity of biological material changes, corresponding volumes and other parameters used in the subprocesses will change as well

In one embodiment of the present invention, there is provided a computer readable medium for controlling the operation of an automated machine, the computer readable medium comprising machine readable instructions for causing a computer to issue a command set capable of causing an automated DNA isolation apparatus to perform a DNA isolation according to the method 200.

FIG. 3 shows another embodiment 300 of the present invention which comprises machine readable instructions stored in a program or module 302 for execution by a computer. Program 302 issues output in the form of commands for controlling the operation of an automated machine for performing the centrifuging, aspirating, dispensing, and mixing operations of the methods 100 and 200. Program modules 304, 306, 308, and 310 within program 302 allow for the custom control of the four basic processes, centrifugation, aspiration, mixing, and dispensing, of DNA isolation according to methods such as methods 100 and 200. It should be understood that different processes of DNA isolation may have different sub-processes, and that such sub-processes are capable of being added to the module 302 without departing from the scope of the invention.

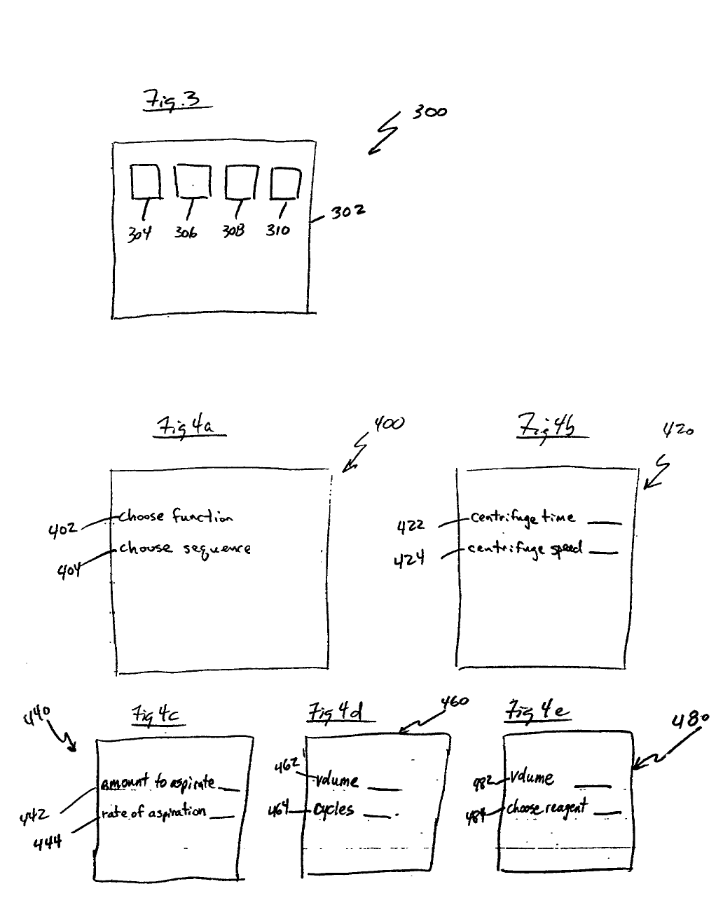

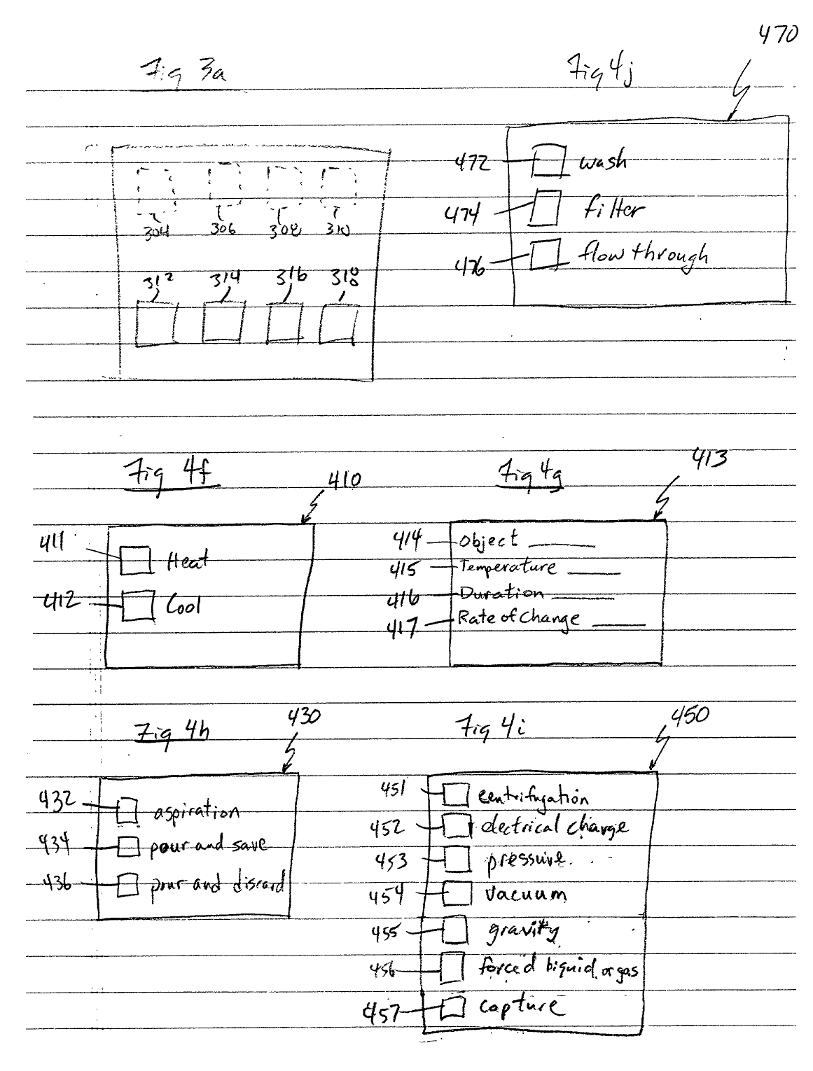

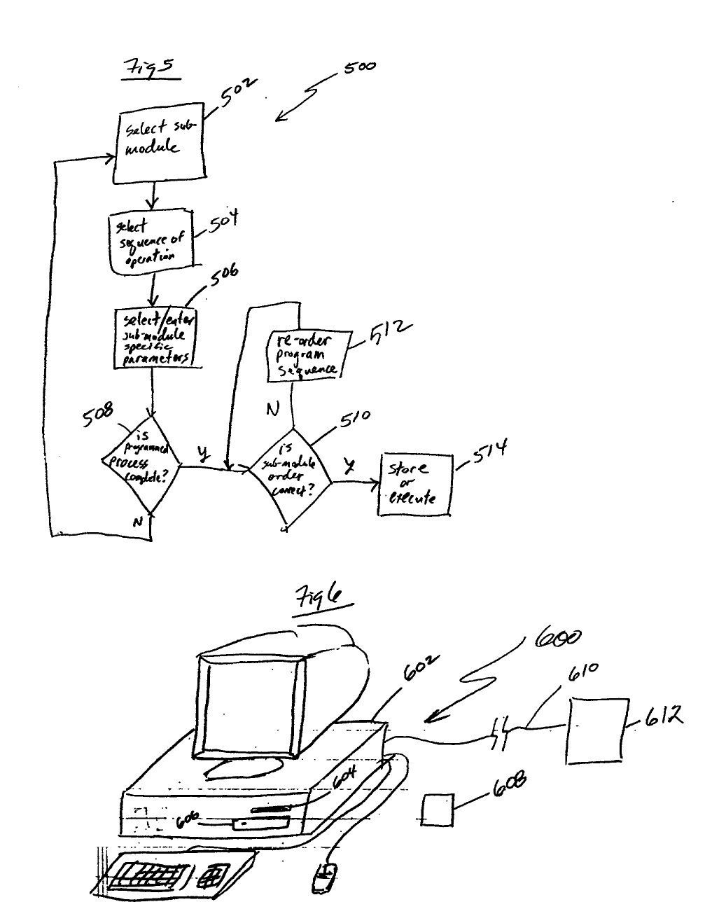

As shown in FIG. 4a, module 302 contains a graphical user interface screen 400 for the construction of a specific combination of process steps generated from a menu of modules 304, 306, 308, and 310. A user may choose the function or module desired at 402, and may choose the sequence of the function at 404. For example, when a user starts module 302, a menu for selecting the first process step appears. Suppose the user wishes to start with a centrifugation step. At 402, the user enters or selects “centrifugation.” At 404, the user enters or selects “1.” When the desired function and order have been entered or selected, a screen representative of the parameters for the chosen operation is displayed.

FIG. 4b shows a representative screen 420 which is displayed when the user has selected to program a centrifuging step. At 422, the user may enter the amount of time the centrifuging should last, and at 424, the user may enter the centrifuge speed. Alternatively, module 304 could be configured so that the entry of a centrifuge speed determined the centrifuge time according to a predetermined ratio of speed to time, or vice versa.

FIG. 4c shows a representative screen 440 which is displayed when the user has selected to program an aspiration step. At 442, the amount of volume to aspirate is entered by the user. At 444, the rate of aspiration maay be entered by the user. Alternatively, aspiration may be accomplished by measuring the amount of material left in the sample container, or material such as supernatant may be removed by weight. Such alternative means by which the correct amount of supernatant may be removed are known in the art, and are within the scope of the invention.

FIG. 4d shows a representative screen 460 which is displayed when the user has selected to program a mixing step. As has been mentioned, mixing may be done in any number of ways, such as by aspirating and dispensing, ultrasonic mixing, agitation, and the like. A representative screen 460 for mixing by aspirating and dispensing allows the user toe enter the volume to aspirate and dispense at 462, and to enter the number of cycles, that is the number of times the aspirating and dispensing is to occur, at 464.

FIG. 4e shows a representative screen 480 which is displayed when the user has selected to program a dispensing step. Various reagents are dispensed in various quantities in a typical DNA isolation process such as those described above in methods 100 and 200. At 482, the user enters the volume of reagent to be dispensed, and at 484 the user enters or selects the specific reagent to dispense.

It is to be understood that the display screens described above may be modified without departing from the scope of the invention.

In another embodiment, program 302 may be used to program the isolation of nucleic acids including DNA and ribonucleic acid (RNA). Further control program modules which are implemented by an automated machine or robotic workstation within program 302 include a temperature control module 312, a material removal module 314, a separation module 316, and a combination removal and separation module 318, shown in block diagram in FIG. 3a.

Temperature control module 312 incorporates the processes of heating and cooling. The heating or cooling of the module 312 may be performed as a standalone step in isolation of nucleic acids. The heating or cooling of the module 312 may also be used in combination with any of the other module processes during an isolation process for nucleic acids. Additionally, the heating or cooling may be performed on samples, remainders of samples, reagents to be dispensed, and the like.

FIG. 4f shows a representative screen 410 which is displayed when the user has selected to program a temperature control step. In module 312, the user can select heating 411 or cooling 412. Once heating 411 or cooling 412 has been selected, a second representative screen 413 shown in FIG. 4g is displayed. From screen 413, the user can choose parameters of the heating or cooling such as the object or sample to be heated or cooled 414, the temperature 415, the duration of the heating or cooling 416, and the rate of temperature change in 417.

Material removal module 314 incorporates processes for removing material from a separated sample, or removing material by volume, weight, mass, and the like. Material removal module 314 includes aspiration as discussed above in conjunction with FIG. 4c. Further material removal processes incorporated into material removal module 314 include pouring material or liquid from a sample. When material is poured from a sample, it is either saved for later use, or discarded.

FIG. 4h shows a representative screen 430 which is displayed when the user has selected to program a material removal step. In module 314, the user selects aspiration 432, pour and save 434, or pour and discard 436.

Separation module 316 incorporates processes for separating out materials within a mixture, colloid, suspension, or the like. Separation module includes centrifugation as discussed above in conjunction with FIG. 4b. Further separation processes incorporated into separation module 316 include electrical charge, pressure, vacuum, gravity, and forced liquid or gas separation processes, and capture including magnetic capture, affinity capture, hybridization capture, electrical capture, molecular bonding, capture by physical size, and the like.

In a forced liquid or gas separation, positive pressure is used to dispense or push gas, reagents, biological liquids, mixtures thereof, and the like through a purification system. Other separations may be performed by liquid phase and/or solid phase chemical means to purify and concentrate analytes such as nucleic acids. Liquid phase methods may use changes in density, electrical charge, temperature, or the like to cause the separation of impurities from the nucleic acids, for example by precipitation. Subsequently, precipitates may be separated by centrifugation, filtration, pouring, or aspiration, among other processes. Precipitates may be either impurities or analyte (such as nucleic acids). Solid phase chemical means may use changes in density, electrical charge, affinity, hybridization, temperature, etc. . . . , to cause impurities or analyte to become attached to or removed from the solid phase. Examples of a solid phases include membranes, rods, mesh, fibers, and particles, with surfaces comprising electrical charge, plastics, silica, cellulose, and the like.

FIG. 4i shows a representative screen 450 which is displayed when the user has selected to program a separation step. In separation module 316, the user selects centrifugation 451, electrical charge 452, pressure 453, vacuum 454, gravity 455, forced liquid or gas 456, and capture 457 from screen 450. Each of the various processes have associated various parameters which may be set using supplemental screens as discussed above.

Combination separation and removal module 318 incorporates processes that have elements of both separation and removal, but which are performed contemporaneously. Separation and removal module 318 includes washing, filtering, and flow through implementations. Washing includes immersing a sample to be washed in a reagent of some sort, passing a reagent over a sample, and the like. Washing may be accomplished with multiple cycles and multiple reagents. Filtering includes sifting a sample through a mesh with a certain pass through size, passing a sample through a mesh having a certain pass through size, and the like. The filtering could result in passing the desired resultant material through the filter or retaining the desired resultant material in the filter.

A flow through purification system involves continuous or discrete binding or trapping an analyte, such as nucleic acids, on a solid phase to separate it from the impurities. During this process the solid phase, having collected the analyte, is washed continuously with reagent or gas carriers to remove the majority of impurities. Conversely, a flow through system with a solid phase binds the impurities and allows the analyte to flow through using reagent or gas carriers with positive or negative pressure.

FIG. 4j shows a representative screen 470 which is displayed when the user has selected to program a removal and separation step. In module 318, the user selects washing 472, filtering 474, or flow through 476 from screen 470. Each of the various processes have associated various parameters which may be set using supplemental screens as discussed above.

The module 302 allows for the coordination of the sequence of the entire process created by selecting a combination of sub-module functions. In this way, a specific process of DNA isolation employing the various sub-processes of the sub-modules 304, 306, 308, and 310 may be controlled by the module 302.

FIG. 5 is a flow chart diagram of a method embodiment 500 for creating a command set for control of an automated DNA isolation apparatus. Method 500 comprises selecting a sub-module for programming in block 502, selecting the order of the specific sub-module execution on the overall program sequence in block 504, and selecting or entering the sub-module specific parameters in block 506. Process flow continues with decision block 508, in which it is determined if the process to be controlled is complete. If the process to control is complete, optional block 510 allows for the determination of whether the control program order is complete. If control program order is not correct, the program sequence is re-ordered in block 512. If the program sequence is correct, the resulting control program is stored or executed in block 514. If the process to be controlled is not complete as determined by decision block 508, the process flow continues with block 502.

By choosing from various sub-modules, and then inputting an execution sequence and parameters for each of the sub-processes controlled my the sub-modules, a complete DNA isolation process is programmable by the method 500. It should be understood that the sequence of method 500 may be altered without departing from the scope of the invention. For example, all of the sub-module operations of a contemplated process could be selected and the parameters entered before any sequencing of the execution of the sub-modules is completed.

As described above, the parameters of the module 302 and sub-modules 304, 306, 308, and 310 may be entered using a graphical user interface of the present invention. When the control operation sequence of the present invention is completed, the operation of the process may be implemented in any machine or apparatus capable of being controlled by the module 302. The module 302 may be tailored to issue command sets readable by any system or apparatus that accepts commands.

Interaction of the operator or technician with the apparatus or system for DNA isolation is substantially reduced by the methods and software modules of the present invention. Accordingly, the risk of contamination, accident, and imprecision in performance of the process steps is reduced. Such an operation could be performed at any time, even overnight, with minimal or even no supervision, provided that the machine or apparatus is capable of performance under unsupervised conditions.

Further control modules may be implemented to allow an automated machine or robotic implementation for DNA isolation to perform even more of the operations normally performed by the technician or operator. For example, a robotic arm could be programmed to select a sample or series of samples from a sample tray or location. Such a tray could be indexed, so that a sub-module such as sub-modules 304, 306, 308, and 310 would allow for the selection of certain of the samples from a tray for DNA isolation according to an embodiment of the methods of the present invention. Samples that have completed the DNA isolation process could similarly be removed from the final process step to an appropriate storage location.

The methods shown in FIG. 5, as well as the module 302 and sub-modules 304, 306, 308, and 310 may be implemented in various embodiments in a machine readable medium comprising machine readable instructions for causing a computer 600 such as is shown in FIG. 6 to perform the methods. The computer programs run on the central processing unit 602 out of main memory, and may be transferred to main memory from permanent storage via disk drive 604 or CD-ROM drive 606 when stored on removable media 608 or via a network connection or modem connection when stored outside of the computer 600, or via other types of computer or machine readable medium from which it can be read and utilized.

Such machine readable medium may include software modules and computer programs. The computer programs comprise multiple modules or objects to perform the methods in FIG. 5, or the functions of various modules in the apparatuses of FIGS. 3, 4a, 4b, 4c, 4d, and 4e. The type of computer programming languages used to write the code may vary between procedural code type languages to object oriented languages. The files or objects need not have a one to one correspondence to the modules or method steps described depending on the desires of the programmer. Further, the method and apparatus may comprise combinations of software, hardware and firmware as is well known to those skilled in the art.

A processor in the computer 600 could have hard-coded (burned into PROM) control software, or it could download the control software from a separate processor or computer system. Computer 600 could be directly controlling an apparatus or machine for DNA isolation, or could be connected via a communication link 610 to a machine or apparatus 612 capable of performing DNA isolation. Communication line 610 may take many forms, such as a parallel or serial communication line, infrared, radio frequency, or other wireless link, and the like.

Further, a microcomputer having a built in processor, onboard RAM, EPROM, and input/output points may be substituted for the computer 600. Also, a programmable logic controller (PLC) may be embodied with the program modules of the present invention. Either the microcomputer or PLC may be operatively connected to an apparatus for isolating DNA, including a stand-alone apparatus or a robotic implementation of a DNA isolation apparatus.

The software implementing the various embodiments of the present invention may be implemented by computer programs of machine-executable instructions written in any number of suitable languages and stored on machine or computer readable media such as disk, diskette, RAM, ROM, or other device commonly included in a personal computer.

It is to be understood that the above description is intended to be illustrative, and not restrictive. Many other embodiments will be apparent to those of skill in the art upon reading and understanding the above description. The scope of the invention should, therefore, be determined with reference to the appended claims, along with the full scope of equivalents to which such claims are entitled.

What is claimed is:

1. A computer readable medium for controlling the operation of an automated machine, the computer readable medium comprising machine readable instructions for causing a computer to perform a method comprising:
issuing a command set to initiate a plurality of nucleic acid isolation functions by a nucleic acid isolation apparatus, wherein the nucleic acid isolation functions comprise:
loading a vessel into a centrifuge;
centrifuging a sample;
aspirating a sample;
mixing a sample;
dispensing into a sample;
controlling the temperature of a function;
removing material from a sample;
separating a sample; and
removing and separating a sample.

2. The computer readable medium of claim 1, wherein controlling the temperature of a function is chosen from a group consisting of heating a sample, cooling a sample, heating a reagent, cooling a reagent, heating while performing a nucleic acid isolation function, and cooling while performing a nucleic acid isolation function.

3. The computer readable medium of claim 1, wherein removing material from sample is done by a method chosen from a group consisting of aspirating, pouring and saving, and pouring and discarding.

4. The computer readable medium of claim 1, wherein separating a sample is done by a method chosen from a group consisting of centrifugation, magnetic capture, electrical charge, gravity, affinity capture, hybridization capture, pressure, vacuum, forced liquid, and forced gas.

5. The computer readable medium of claim 1, wherein removing and separating a sample is done by a method chosen from the group consisting of washing, filtering, and flow through.

6. A computer system for configuring a machine to automatically perform a method of isolating nucleic acids, the computer system comprising:
a computer;
a computer readable medium comprising machine readable instructions for causing the computer to output a command series to an automated nucleic acid isolation machine for control of the functions of nucleic acids isolation process.

7. The computer system of claim 6, wherein the computer readable medium comprises:
a software module comprising:
a centrifugation sub-module for issuing commands initiating centrifuging of a sample for a centrifuge time and a centrifuge speed;
an aspiration sub-module for issuing commands initiating aspirating a sample to remove a volume of fluid from a sample;
a mixing sub-module for issuing commands initiating mixing a sample;
a dispensing module for issuing commands initiating dispensing into a sample an amount of a specific reagent;
a temperature control module for issuing commands to control the temperature of a function;
a removal module for issuing commands to remove material from a sample;
a separation module for issuing commands to separate a sample into components; and
a combination removal and separation module for issuing commands to control separating and removing a sample.

8. The computer system of claim 6, and further comprising:
a graphical user interface for selecting a sequence of commands to be output by the computer.

9. A control module for controlling the operation of an automated nucleic acids isolation apparatus, the module comprising:
a processor; and
a program module comprising a set of machine readable instructions for issuing commands to the automated nucleic acids isolation apparatus to perform a series of steps, comprising:
centrifuging a sample;
aspirating a sample;
mixing a sample;
adding a reagent to the sample;
controlling the temperature of an isolation function;
removing material from a sample;
separating a sample; and
separating and removing a sample.

10. The control module of claim 9, wherein the control module is implemented in a computer readable medium.

11. The control module of claim 9, wherein the control module is implemented in a dedicated processor.

12. The control module of claim 9, wherein the program module is burned into the processor in hard code.

13. The control module of claim 9, wherein the program module is implemented in a programmable logic controller.

14. A computer control module for an automated nucleic acids isolation apparatus, the control module comprising:
a plurality of sub-modules, each sub-module comprising machine readable instructions for creating a command to the nucleic acids isolation apparatus to perform a process step of the nucleic acids isolation process; and
an output link for communicating the commands to the nucleic acids isolation apparatus.

15. The computer module of claim 14, wherein the plurality of sub-modules comprises:
a centrifuge sub-module for issuing commands initiating centrifuging of a sample for a centrifuge time and a centrifuge speed;
an aspirate sub-module for issuing commands initiating aspirating a sample to remove a volume of fluid from a sample;
a mixing sub-module for issuing commands initiating mixing a sample;
a dispensing module for issuing commands initiating dispensing into a sample an amount of a specific reagent;
a temperature control module for issuing commands to control the temperature of a function;
a removal module for issuing commands to remove material from a sample;
a separation module for issuing commands to separate a sample into components; and
a combination removal and separation module for issuing commands to control separating and removing a sample.

16. The computer module of claim 14, and further comprising
a user input/output interface for programming a process comprising a plurality of invocations of the various sub-modules of the computer module.

17. The computer module of claim 14, wherein each of the sub-modules is configured to accept input of values for issuing commands.

18. The computer control module of claim 17, wherein the control module is implemented in a machine readable medium comprising a set of machine readable instructions.

19. The computer module of claim 14, wherein the control module is implemented in a dedicated processor.

20. A method of defining a protocol for automated isolation of nucleic acids by an apparatus for nucleic acids isolation using a software module having a plurality of nucleic acids isolation sub-modules, the method comprising:
selecting a sub-module;
selecting an operational sequence for the selected sub-module;
defining the sub-module specific parameters; and
repeating selecting a sub-module through defining the sub-module parameters until the desired protocol is complete.

21. The method of claim 20, and further comprising:
re-ordering the sub-module execution sequence after the desired protocol is complete.

22. The method of claim 20, wherein the method may be performed in a different order.
